(19) United States
(12) Patent Application Publication
(10) Pub. No.: US 2007/0163584 A1
(43) Pub. Date: Jul. 19, 2007

(54) Method and apparatus for controlling at least one ventilation parameter of an artificial ventilator for ventilating the lung of a patient in accordance with a plurality of lung positions

(75) Inventors: Stephan Bohm, Hamburg (DE); Royce W. Johnson, Universal City, TX (US)

Correspondence Address: LEGAL DEPARTMENT INTELLECTUAL PROPERTY;KINETIC CONCEPTS, INC., P.O. BOX 659508, SAN ANTONIO, TX, 78265-9508, US

(73) Assignee: KCI LICENSING, INC., San Antonio, TX (US)

(21) Appl. No.: 10/594,400

(22) PCT Filed: Mar. 29, 2005
(86) PCT No.: PCT/US05/10741

§ 371 Date: Sep. 26, 2006

(30) Foreign Application Priority Data
Mar. 29, 2004 (EP) 04 007 580.6
Mar. 11, 2005 (EP) 05 005 418.8

(51) Int. Cl. A61M 16/00; A62B 7/00

(52) U.S. Cl. 128/204.18; 128/200.24; 128/204.21

(57) ABSTRACT

The invention refers to a method and an apparatus for controlling at least one ventilation pressure of an artificial ventilator for ventilating an artificially ventilated lung of a patient in accordance with a plurality of lung positions. In order to improve the potentials of the kinetic rotation therapy, at least one ventilation pressure is controlled in accordance with a defined lung position and in accordance with a lung status information related to said defined lung position.

The invention refers to a method and apparatus for recording the status of an artificially ventilated lung of a patient in accordance with a plurality of lung positions and to a method and apparatus for controlling at least one ventilation parameter of an artificial ventilator for ventilating an artificially ventilated lung of a patient in accordance with a plurality of lung positions. Furthermore, the invention refers to a method and an apparatus for controlling the change of the position of an artificially ventilated lung of a patient. For carrying out the invention it is assumed that the patient lies in a nursing bed and that the position of the artificially ventilated lung is movable or changeable by a position actuator. An example for such a nursing bed is a rotation bed which is rotatable by a rotation angle around its longitudinal axis.

The treatment of acute lung failure, acute lung injury (ALI) and acute respiratory distress syndrome (ARDS) is still one of the key problems in the treatment of severely ill patients in the intensive care unit. Despite intensive research over the past two decades the negative implications of respiratory insufficiency are still affecting both the short and long term outcome of the patient. While different ventilator strategies have been designed to treat the oxygenation disorder and to protect the lungs from ventilator induced lung injury, additional therapeutic options were evaluated.

Dynamic body positioning (kinetic or axial rotation therapy) was first described by Bryan in 1974. This technique is known to open atelectasis and to improve lung function, particularly arterial oxygenation in patients with ALI and ARDS. Since kinetic rotation therapy is a non-invasive and relatively inexpensive method it can even be used prophylactically in patients whose overall health condition or severity of injury predispose to lung injury and ARDS. It could be shown that the rate of pneumonia and pulmonary complications can be reduced while survival increased if kinetic rotation therapy is started early on in the course of a ventilator treatment. This therapeutic approach may reduce the invasiveness of mechanical ventilation (i.e. airway pressures and tidal volumes), the time on mechanical ventilation and the length of stay on an intensive care unit.

Kinetic rotation therapy in the sense of the present invention is applied by use of specialized rotation beds which can be used in a continuous or a discontinuous mode with rests at any desired angle for a predetermined period of time. The general effect of axial rotation in respiratory insufficiency is the redistribution and mobilization of both intra-bronchial fluid (mucus) and interstitial fluid from the lower (dependent) to the upper (non-dependent) lung areas which will finally lead to an improved matching of local ventilation and perfusion. As a consequence, oxygenation increases while intra-pulmonary shunt decreases. Lymph flow from the thorax is enhanced by rotating the patient. In addition, kinetic rotation therapy promotes the recruitment of previously collapsed lung areas, thus reducing the amount of atelectasis, at identical or even lower airway pressures. At the same time now-opened lung areas are protected from the shear stress typically caused by the repetitive opening and closing of collapse-prone alveoli in the dependent lung zones.

From H. C. Pape, et al.: “Is early kinetic positioning beneficial for pulmonary function in multiple trauma patients?”, Injury, Vol. 29, No. 3, pp. 219-225, 1998 it is known to use the kinetic rotation therapy which involves a continuous axial rotation of the patient on a rotation bed. It has been found that the kinetic rotation therapy improves the oxygenation in patients with impaired pulmonary function and with post-traumatic pulmonary insufficiency and adult respiratory distress syndrome (ARDS).

However, since the kinetic rotation therapy requires a specially designed rotation bed it has not been found yet that the kinetic rotation therapy justifies a broad employment. Further, kinetic rotation therapy has been utilized with standardized treatment parameters, typically equal rotation from greater than 45 degrees to one side to greater than 45 degrees to the other side, and 15 minute cycle times. These rotation parameters are rarely altered in practise due to a lack of conjoint ventilation effectiveness and rotation activity information. Similarly, the lack of conjoint information hampers practitioners from taking advantage of the beneficial effects of kinetic rotation therapy by reducing the aggressiveness of mechanical ventilation parameters employed to treat a rotated patient.

It is an object of the invention to improve the potentials of the kinetic rotation therapy.

This object is solved according to a first inventive solution by a recording method for recording the status of an artificially ventilated lung of a patient in accordance with a plurality of lung positions, the patient lying in a nursing bed and the position of the artificially ventilated lung is movable by a position actuator, comprising the steps of:
a) moving the artificially ventilated lung by the position actuator to a defined lung position,
b) determining the status of the artificially ventilated lung, and
c) recording the status of the artificially ventilated lung in accordance with the defined lung position.

A corresponding recording apparatus according to the first inventive solution for recording the status of an artificially ventilated lung of a patient lying in a nursing bed in accordance with a plurality of lung positions comprises the following features:
a) a position actuator for moving the artificially ventilated lung to a defined lung position,
b) determining means for determining the status of the artificially ventilated lung, and
c) recording means for recording the status of the artificially ventilated lung in accordance with the defined lung position.

The first inventive solution is based on the cognition that the change of the lung position of an artificially ventilated lung also changes the status of the artificially ventilated lung. Therefore, a reproducible recording of the status of the artificially ventilated lung in accordance with the defined lung position is carried out which enables a purposeful treatment of the lung by other means.

Furthermore, the object is solved according to a second inventive solution by a controlling method for controlling at least one ventilation parameter of an artificial ventilator for ventilating an artificially ventilated lung of a patient in accordance with a plurality of lung positions, the patient lying in a nursing bed and the position of the artificially ventilated lung is movable by a position actuator, comprising the steps of:
a) obtaining lung status information which is based on at least two supporting points of a first status of the artificially ventilated lung in accordance with a first lung position and a second status of the artificially ventilated lung in accordance with a second lung position,
b) moving the artificially ventilated lung by the position actuator to a defined lung position,
c) controlling of at least one ventilation parameter in accordance with the defined lung position and in accordance with the lung status information related to said defined lung position.

A corresponding controlling apparatus according to the second inventive solution for controlling at least one ventilation parameter of an artificial ventilator for ventilating an artificially ventilated lung of a patient lying in a nursing bed in accordance with a plurality of lung positions comprises the features of:
a) means for obtaining lung status information which is based on at least two supporting points of a first status of the artificially ventilated lung in accordance with a first lung position and a second status of the artificially ventilated lung in accordance with a second lung position,
b) a position actuator for moving the artificially ventilated lung to a defined lung position,
c) means for controlling of at least one ventilation parameter in accordance with the defined lung position and in accordance with the lung status information related to said defined lung position.

The second inventive solution is based on the cognition that the change of the lung position of an artificially ventilated lung also changes the status of the artificially ventilated lung which can be used for an optimized ventilation. Thereby, the already known kinetic rotation therapy can be supported. More particularly, an optimized ventilation according to the second inventive solution considers the fact that the top positioned lung during the rotation therapy is relieved from superimposed pressures. For example, in order to reach the optimum of at least one ventilation pressure during rotation, at least a second status of the artificially ventilated lung is determined and is compared with a previously determined first status of the artificially ventilated lung, wherein at least one ventilation pressure is controlled in accordance with the difference between the first status and the second status of the artificially ventilated lung.

Furthermore, the object is solved according to a third inventive solution by a positioning method for controlling the change of the position of an artificially ventilated lung of a patient, the patient lying in a nursing bed and the position of the artificially ventilated lung is changeable by a corresponding position actuator, comprising the steps of:
a) providing a periodical controlling signal having a distribution of a plurality of position periods and/or of a plurality of amplitudes,
b) controlling the position actuator by said periodical controlling signal.

A corresponding positioning apparatus according to the third inventive solution for controlling the change of the position of an artificially ventilated lung of a patient lying in a nursing bed comprises the features of:
a) a position actuator for changing the position of the artificially ventilated lung,
b) means for providing a periodical controlling signal having a distribution of a plurality of position periods and/or of a plurality of amplitudes, and
c) means for controlling the position actuator by said periodical controlling signal.

The third inventive solution is based on the cognition that the parameters of the controlling signal which controls the position actuator and thereby the lung position influences also the success of the kinetic rotation therapy. An important parameter is the rotation period or the movement period which is the period of time in which the lung position returns after a movement in one direction back to its starting position. A further cognition of the third inventive solution is the fact that the success of the kinetic rotation therapy can be improved if the rotation period and/or the rotation amplitude is not fixed but varies statistically around a predetermined mean rotation period.

The first inventive solution, the second inventive solution and the third inventive solution can be combined with each other. The preferred aspects described in the following can be applied to each of the inventive solutions.

According to one aspect, the nursing bed is rotatable around its longitudinal axis and the position actuator is a motor rotating the nursing bed around its longitudinal axis. Alternatively, it is also possible that the position actuator comprises air-filled or fluid-filled cushions provided underneath the patient.

According to a further aspect, the defined lung position is reached by a predetermined step size of the position actuator. Alternatively, it is also possible that the defined lung position is reached in accordance with a feed back signal of a position sensor measuring the actual lung position.

According to a further aspect, the status of the artificially ventilated lung is a measure of a regional or a global information on lung morphology and/or lung function.

Regional information enables a specific treatment of a part of the lung and can be realized by imaging methods, like the electrical impedance tomography (EIT) or computed tomography (CT). Global information of the lung are easier to obtain, e.g. by the measurement of gas exchange, but measure merely the behavior of the whole lung.

The lung morphology considers structural features of the lung, i.e. the anatomy and its abnormalities whereas the lung function refers to the dynamic behaviour like ventilation and blood flow as well as to the mechanical behaviour of the lung.

According to a preferred aspect, the status of the artificially ventilated lung is a measure of the functionality with regard to the global gas exchange of the lung. There are multiple methods and apparatuses for determining global gas exchange of which some are mentioned in the following.

The status of the lung can be determined on the basis of the CO₂ concentration of the expired gas over a single breath. Such a method and apparatus are known from the previous European patent application “Non-Invasive Method and Apparatus for Optimizing the Respiration for Atelectatic Lungs”, filed on 26 Mar. 2004, which is herewith incorporated by reference.

Furthermore, the status of the lung can be determined on the basis of the hemoglobin oxygen saturation (SO₂). This can be carried out by means of a saturation sensor. Advantageously, a feedback control loop controls the inspiratory oxygen fraction (FiO₂) at the artificial ventilator such that the hemoglobin oxygen saturation (SO₂) is kept constant and a data processor determines during a change of the airway pressure from the course of the controlled inspiratory oxygen fraction (FiO₂) an airway pressure level which corresponds to alveolar opening or alveolar closing of the lung. Such a method and apparatus are known from WO 00/44427 A1 which is herewith incorporated by reference.

Furthermore, the status of the lung can be determined on the basis of the CO₂ volume exhaled per unit time. Such a method and apparatus are known from WO 00/44427 A1 which is herewith incorporated by reference.

Furthermore, the status of the lung can be determined on the basis of the endtidal CO₂ concentration. Such a method and apparatus are known from WO 00/44427 A1 which is herewith incorporated by reference. Furthermore, the status of the lung can be determined on the basis of the arterial partial pressures of oxygen paO₂. Such a method and apparatus are known from S. Leonhardt et al.: “Optimierung der Beatmung beim akuten Lungenversagen durch Identifikation physiologischer Kenngröβen”, at 11/98, pp. 532-539, 1998 which is herewith incorporated by reference.

According to a further aspect, the status of the lung can be determined on the basis of the compliance of the lung, wherein the compliance can be defined by the tidal volume divided by the pressure difference between peak inspiratory pressure and positive end-expiratory pressure (PIP-PEEP). Definitions of the compliance are known e.g. from WO 00/44427 A1 which is herewith incorporated by reference.

According to a further aspect, the status of the lung can be determined on the basis of the inspiratory and/or expiratory dynamic airway resistance, wherein these resistances can be defined as the driving pressure difference divided by the flow of breathing gases (cmH₂0/l/s). Definitions of the resistance are known e.g. from WO 00/44427 A1 which is herewith incorporated by reference.

According to a further aspect, the determined status of the lung is sensitive to changes of alveolar dead space. The aim is to compensate the changes of alveolar dead space by a suitable adjustment of the positive end-expiratory pressure (PEEP) and peak inspiratory pressure (PIP). Various methods and apparatuses are known for determining changes of alveolar dead space of an artificially ventilated lung which can be used separately or in combination with each other.

According to a further aspect, the status of the lung is determined on the basis of electrical impedance tomography data. Such a method and apparatus are known from WO 00/33733 A1 and WO 01/93760 A1 which are herewith incorporated by reference.

Furthermore, many other known clinical methods and apparatuses of assessment of lung function, which may combine both gas exchange effects and hemodynamic efficiency measures, may be employed to determine the status of the artificially ventilated lung. Several of these include pulmonary shunt fraction, oxygen extraction ratio, extravascular lung water, pulmonary vascular resistance and compliance, and the like.

Furthermore, many other known clinical methods and apparati of assessment of lung recruitment and mechanical function may be employed to determine the status of the artificially ventilated lung. These include upper and lower inflection points of the expiratory and inspiratory pressure-volume curves, the point of maximal pressure-volume compliance (Pmax), and others.

According to a further aspect, the determined status of the artificially ventilated lung is recorded by a computer in accordance with the corresponding defined lung position. Preferably, the recorded data are displayed accordingly on a screen.

The recording method and the recording apparatus according to the first inventive solution can be used to provide a lung status information for the controlling method and the controlling apparatus according to the second inventive solution and for the positioning method and the positioning apparatus according to the third inventive solution.

According to one aspect, a predetermined differential step size is applied repeatedly to the position actuator to obtain after each differential step size a supporting point of the status of the artificially ventilated lung until such supporting points of the status of the artificially ventilated lung have been determined over a predetermined range of lung positions.

In order to increase the resolution of the supporting points, the lung status information can be interpolated between the supporting points in accordance with the difference between two neighbouring supporting points. Other interpolating methods may be used which are based on more than two supporting points, e.g. the least square method, by which a steady curve of the lung status information can be obtained over the predetermined range of lung positions.

The obtained lung status information can be used to optimize at least one ventilation parameter of the artificially ventilated lung over the predetermined range of lung positions according to the second inventive solution. Preferably, at least one ventilation parameter is controlled such that the lung status information yields a homogeneous distribution over the predetermined range of lung positions. Thereby, the deviations of the lung status information over the predetermined range of lung positions can be levelled out by applying the appropriate ventilation parameter in accordance with the corresponding lung position. Alternatively, a single ventilation parameter value may be determined from the steady curve to insure maximum lung function as determined by the lung status information over the range of lung positions.

According to a further aspect, at least one ventilation parameter can be controlled such that the determined changes of alveolar dead space are compensated according to the difference between two supporting points of the lung status information of the artificially ventilated lung. For this purpose, a characteristic curve can be recorded for the corresponding lung showing the relationship between alveolar dead space on the one hand and the influence of peak inspiratory pressure (PIP) and positive end-expiratory pressure (PEEP) thereon on the other hand. Based on this characteristic curve the peak inspiratory pressure (PIP) and/or positive end-expiratory pressure (PEEP) can be determined for compensating any changes in alveolar dead space. In order to consider additionally the rotation angle by the characteristic curve, the status of alveolar dead space vs. PIP and/or PEEP is determined in accordance with the plurality of lung positions.

The obtained lung status information can also be used to optimize the controlled change of the position of an artificially ventilated lung according to the third inventive solution. According to the third inventive solution, a distribution of a plurality of position periods and/or of a plurality of amplitudes has to be provided. This can be carried out automatically on the basis of the lung status information which is based on at least two supporting points of a first status of the artificially ventilated lung in accordance with a first lung position and a second status of the artificially ventilated lung in accordance with a second lung position. For example, a look-up table can be provided which assigns for a specific lung status information a corresponding control signal for the position actuator having a specific position period and a specific position amplitude. Thereby, the controlling signal for the position actuator is made up of a plurality of curve pieces over the predetermined range of lung positions which yields over time a distribution of position periods and/or amplitudes.

Alternatively, the distribution can be compiled via a user's interface on the basis of a given set of periodical controlling signals for providing a predetermined distribution.

Alternatively, the distribution can be compiled automatically in advance or online and can follow a known probability distribution or can follow a biologic variability. For example, the human's heartbeat follows a characteristic biologic variability which can be scaled and adapted to provide for the described purpose.

Other objects and features of the invention will become apparent by reference to the following specifications, in which

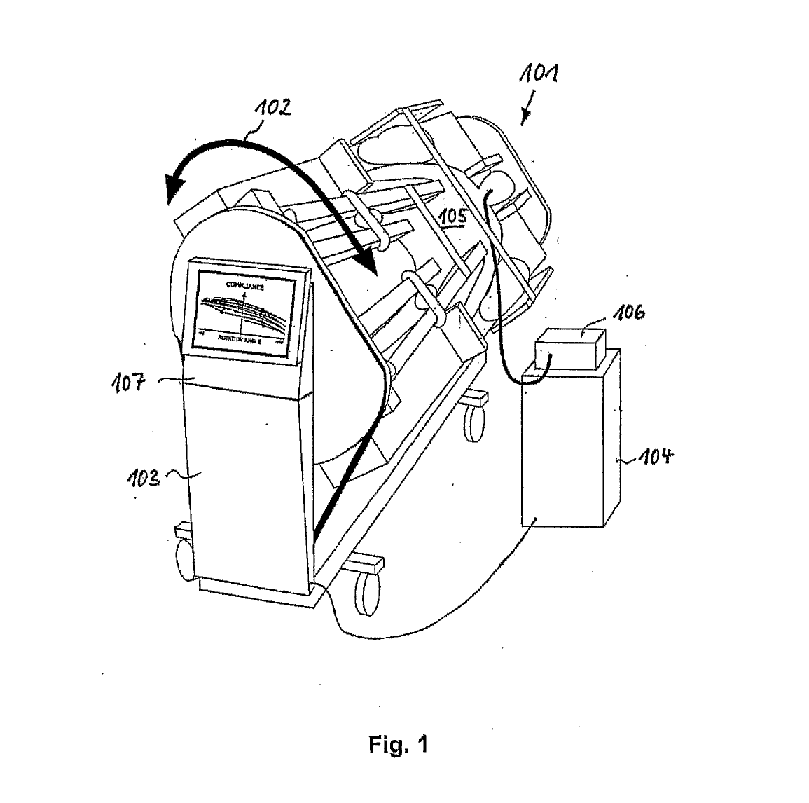

FIG. 1 shows an example of a nursing bed according to the invention,

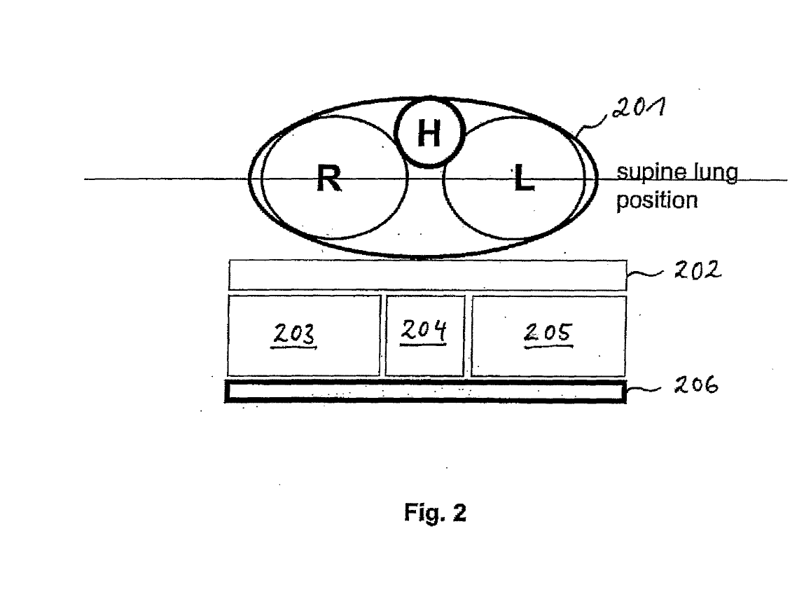

FIG. 2 shows a first example of a position actuator in a horizontal position,

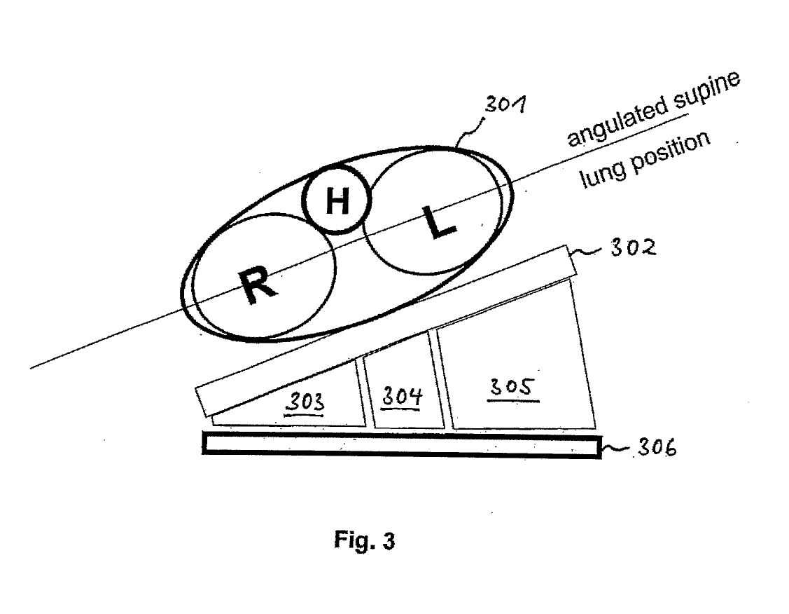

FIG. 3 shows the first example of a position actuator in an angulated position,

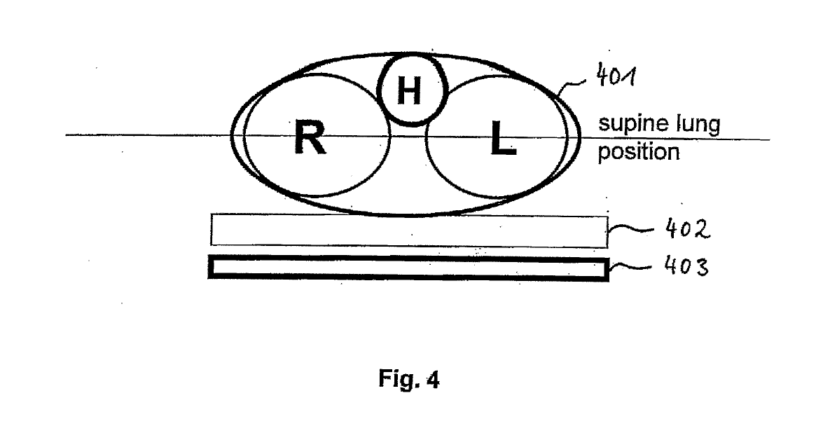

FIG. 4 shows a second example of a position actuator in a horizontal position,

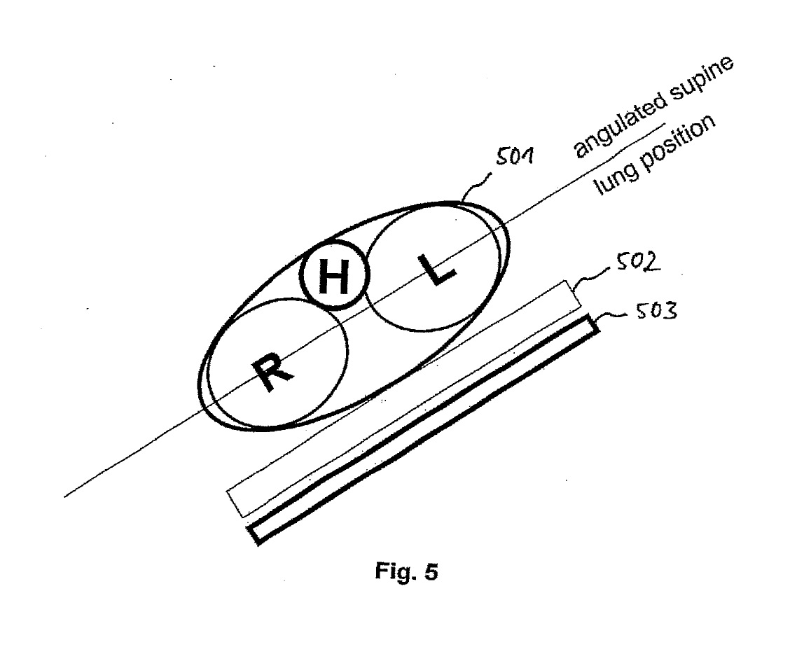

FIG. 5 shows the second example of a position actuator in an angulated position,

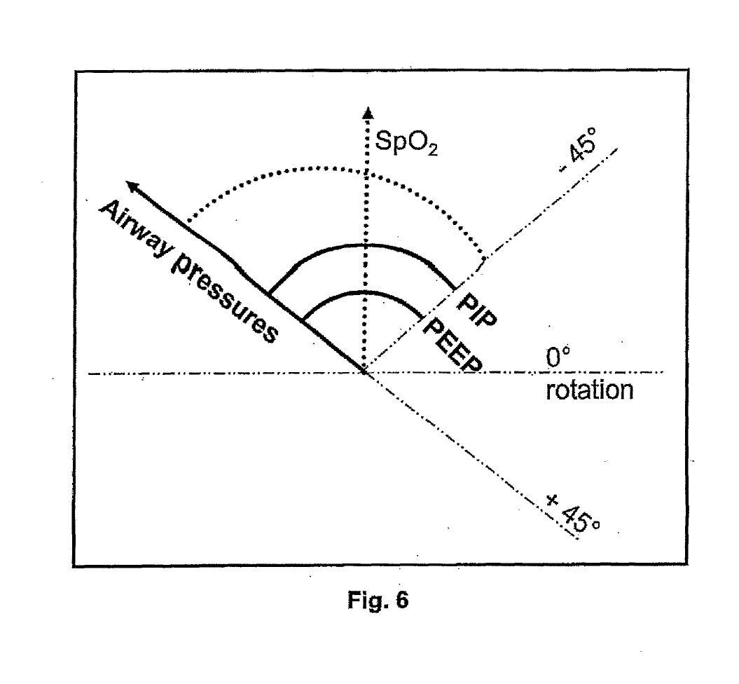

FIG. 6 shows a schematic monitoring screen for the method for controlling at least one ventilation pressure,

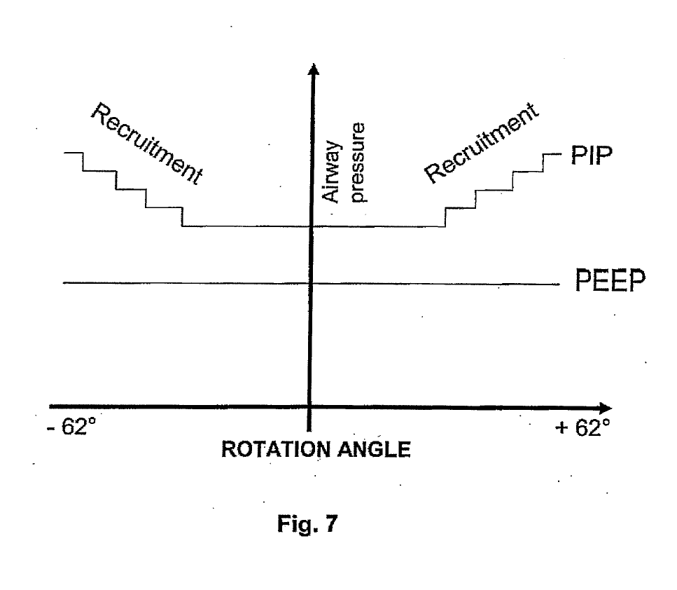

FIG. 7 shows an alveolar recruitment maneuver during kinetic rotation therapy,

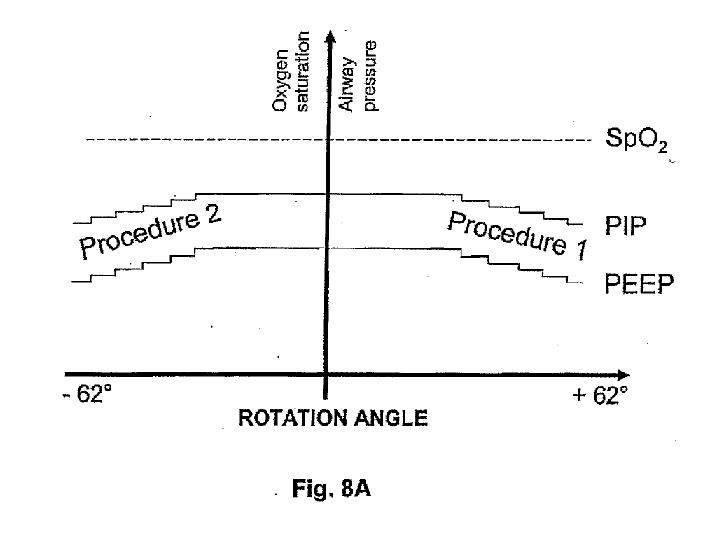

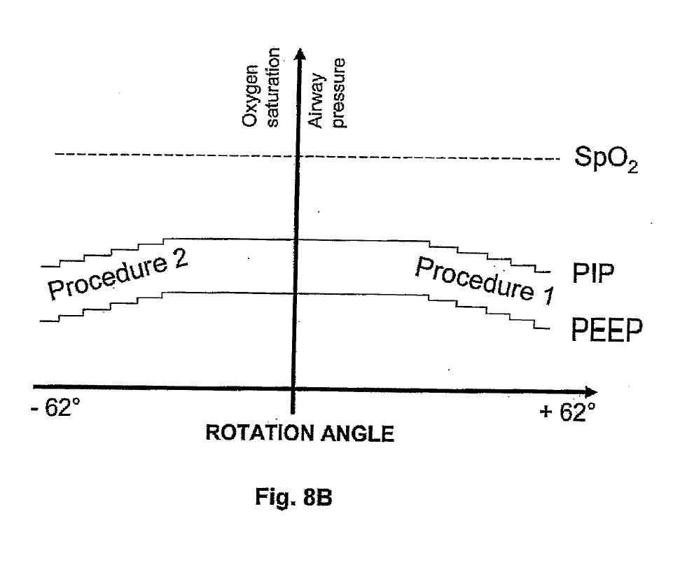

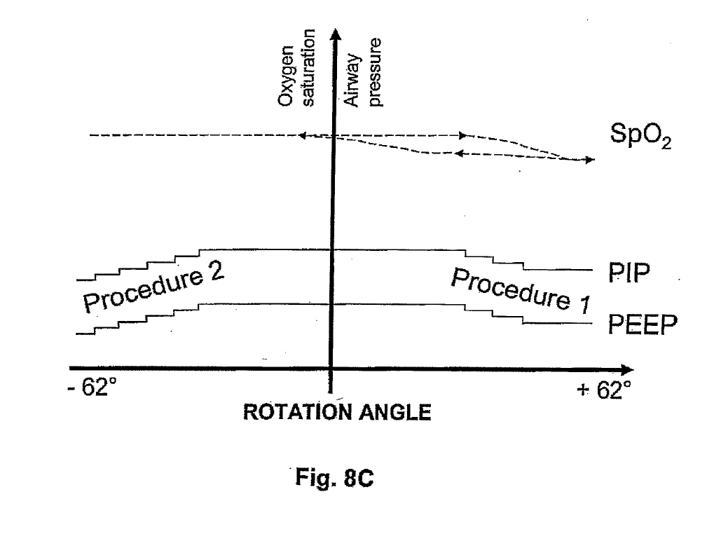

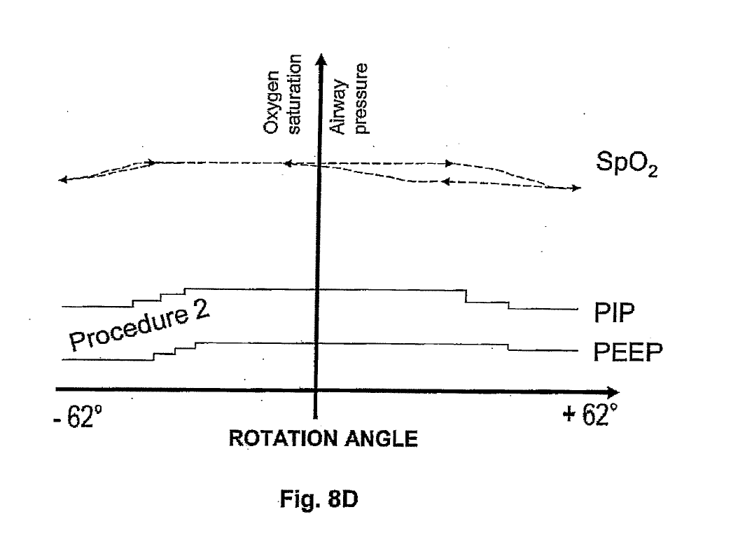

FIG. 8 shows the titration process after a successful lung recruitment maneuver has been performed during kinetic rotation therapy,

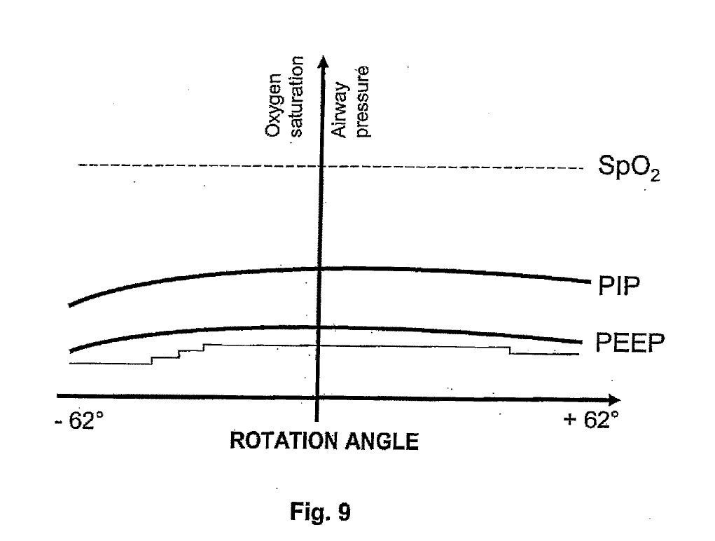

FIG. 9 shows an artificial ventilation of a lung by controlling the PIP and the PEEP in accordance with the rotation angle,

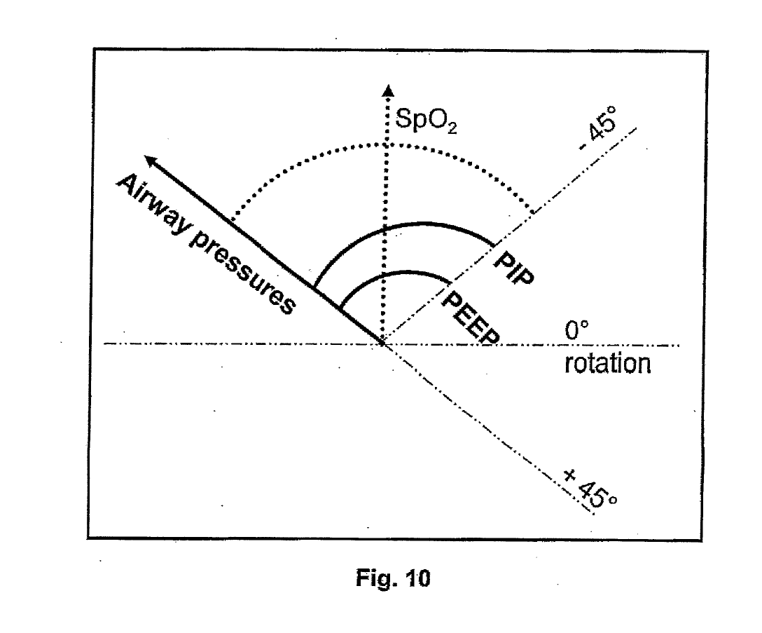

FIG. 10 shows a schematic monitoring screen when controlling the PIP and PEEP during the rotation cycle according to FIG. 9,

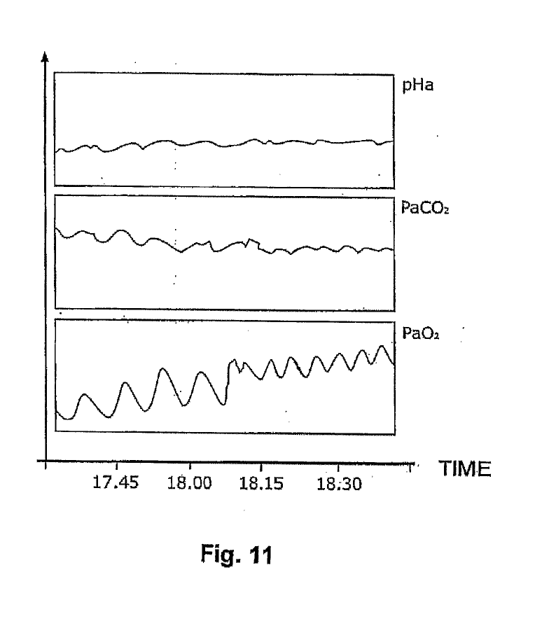

FIG. 11 shows the measurements of paO₂, paCO₂, and pHa during the kinetic rotation therapy, and

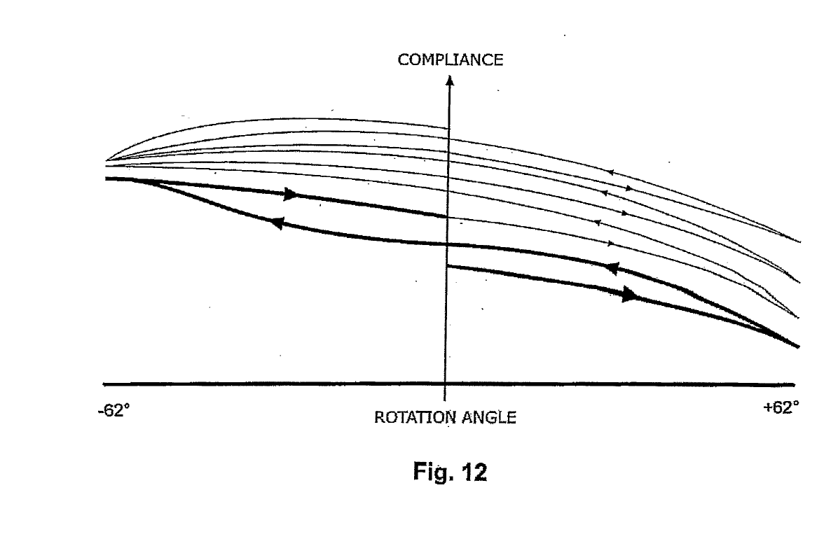

FIG. 12 shows the measurement of compliance during kinetic rotation therapy.

FIG. 1 shows an example of a nursing bed according to the invention. The nursing bed 101 is mounted such that it can be rotated around its longitudinal axis, as indicated by the arrow 102. The rotation angle is changeable by a position actuator 103, which is controlled by a control unit 104.

The patient 105 is fixed on the nursing bed 101 and is artificially ventilated by the ventilator 106. The position actuator 103 can be controlled by the control unit 104 such that the patient is turned resulting in a defined lung position of the artificially ventilated lung. The lung position refers to the rotation angle of the lung being 0° if the patient is lying horizontally on the bed, which itself is positioned horizontally. Measurements of the lung position can be performed by employing a portable position sensor attached to the patient's thorax and connected to the control unit 104. The nursing bed 101 shown in FIG. 1 allows also to determine the rotation angle of the patient's lung through a measurement of the rotation angle of the nursing bed 101.

The status of the artificially ventilated lung can be determined by a variety of methods using a suitable measurement device 107. The measurement device 107 can for example use data such as airway pressures, constitution of the expired gas, and the volume of the inspired and expired gas obtained from the artificial ventilator to determine the status of the lung. The measurements to determine the status of the lung can either be performed continuously or sporadically at defined lung positions. Examples of methods to determine the status of the lung are given below:
The status of the lung is determined on the basis of the CO₂ concentration of the expired gas over a single breath. Such a method and apparatus are known from the European patent application “Non-Invasive Method and Apparatus for Optimizing the Respiration for Atelectatic Lungs”, filed on 26 Mar. 2004, which is herewith incorporated by reference.
The status of the lung is determined on the basis of the hemoglobin oxygen saturation (SO₂). This can be carried out by means of a saturation sensor. Advantageously, a feedback control loop controls the inspiratory oxygen fraction (FiO₂) at the artificial ventilator such that the hemoglobin oxygen saturation (SO₂) is kept constant and a data processor determines during a change of the airway pressure from the course of the controlled inspiratory oxygen fraction (FiO₂) an airway pressure level which corresponds to alveolar opening or alveolar closing of the lung. Such a method and apparatus are known from WO 00/44427 A1 which is herewith incorporated by reference.
The status of the lung is determined on the basis of the CO₂ volume exhaled per unit time. Such a method and apparatus are known from WO 00/44427 A1 which is herewith incorporated by reference.
The status of the lung is determined on the basis of the endtidal CO₂ concentration. Such a method and apparatus are known from WO 00/44427 A1 which is herewith incorporated by reference.
The status of the lung is determined on the basis of the arterial partial pressures of oxygen paO₂. Such a method and apparatus are known from S. Leonhardt et al.: “Optimierung der Beatmung beim akuten Lungenversagen durch Identifikation physiologischer Kenngröβen”, at 11/98, pp. 532-539, 1998 which is herewith incorporated by reference.
The status of the lung is determined on the basis of the compliance of the lung, wherein the compliance can be defined by the tidal volume divided by the pressure difference between peak inspiratory pressure and positive end-expiratory pressure (PIP-PEEP). Definitions of the compliance are known e.g. from WO 00/44427 A1 which is herewith incorporated by reference.
The status of the lung is determined on the basis of the inspiratory and/or expiratory dynamic airway resistance, wherein these resistances can be defined as the driving pressure difference divided by the flow of breathing gases (cmH₂0/l/s). Definitions of the resistance are known e.g. from WO 00/44427 A1 which is herewith incorporated by reference.
The status of the lung is determined on the basis of electrical impedance tomography data. Such a method and apparatus are known from WO 00/33733 A1 and WO 01/93760 A1 which are herewith incorporated by reference.

In the following, an example of a treatment of the patient will be described which will be explained thereafter in more detail by means of the FIGS. 2-12.

Recruitment Maneuver

At 0° rotation angle PEEP is adjusted above the expected alveolar closing pressure (depending on the lung disease between 15 and 25 cmH₂O). PIP is set sufficiently high above PEEP to ensure adequate ventilation.

Then rotation is started. Each lung is opened separately while it is moved into the upward position.

With increasing rotation angle, a stepwise increase of the PIP starts 5-20 breaths prior to reaching the maximum rotation angle, PIP reaches its maximum value (depending on the lung disease between 45 and 65 cmH₂O) at the maximum rotation angle.

Having crossed the maximum rotation angle PIP is decreased within 5-20 breaths.

After each lung has been recruited separately (by rotating the patient to both sides) in the above manner, PIP is adjusted for each lung separately to maintain adequate ventilation.

PEEP Titration for Finding the Closing PEEP

After a recruitment maneuver, PEEP is decreased continuously with increasing rotation angles. The status of the artificially ventilated lung is recorded continuously. Starting at a given PEEP at a rotation angle of 0°, PEEP will be lowered such that at maximum rotation angle PEEP will be reduced by 1-2 cmH₂O (procedure 1). If no signs for alveolar collapse occur in any of the above signals the level of PEEP is recorded and will be increased continuously to the previous setting when at 0°. While turning the patient to the other side PEEP is reduced in the same way (procedure 2). If no signs for alveolar collapse occur in any of the above signals, the level of PEEP is then kept at this value and the patient is turned back to 0°.

If no collapse is present at a rotation angle of 0° the procedures 1 and 2 are carried out at reduced PEEP levels until signs of alveolar collapse occur. The level of PEEP at which this collapse occurs is then recorded for the respective side. The PEEP will be increased continuously to the previous setting when at 0° while turning the patient back to 0°. If due to a hysteresis behaviour of the lung signs of a lung collapse are still present, a recruitment maneuver will be performed at this stage to re-open the lung as described above.

Continuing with an open lung condition, the PEEP is set 2 cmH₂O above the known closing pressure for the side for which the lung collapse occurred.

Thereafter, PEEP is reduced in the way described above while turning the patient to the opposite side for which the closing pressure is not yet known. Once collapse occurs also for this side, PEEP is recorded and the lung is reopened again.

Controlling the Ventilation Parameters During Rotation

After having determined the PEEP collapse pressure of each side, PEEP will be adjusted continuously with the ongoing rotation while making sure that PEEP never falls below the levels needed for each one of the sides.

Since PEEP and compliance may vary with the rotation angle adjustments are needed. Therefore, during rotation therapy PIP levels are adjusted continuously from breath to breath in accordance with the difference between a first status and a second status of the artificially ventilated lung in order to ventilate the patient sufficiently while keeping tidal volumes within a desired range of 6-10 ml/kg body weight.

Furthermore, if PIP pressures are at very low values already, it might be advisable to leave PIP constant but adjust for changes in compliance by adjusting the respiratory rate (RR). Then, RR is adjusted continuously from breath to breath in order to ventilate the patient sufficiently while keeping PIP constant.

It has been shown that the variation of the rotation period improves the effect of the kinetic rotation therapy even further. For example, the following modes of variation can be applied:
Sinusoidal variation with wave length between several minutes to several hours with set minimum and maximum values for ration angles, speeds and resting periods.
Ramp like variation within certain boundaries with ramp periods between several minutes to several hours and set minimum and maximum values for rotation angles, speeds and resting periods.
Random variation about a given mean value at a single level of variability (i.e. biologic variability) with amplitudes between 50% to 200% of mean sequence of magnitude of this parameter from a uniform probability distribution between e.g. 0% to 100% of its chosen mean value.
Variability can be determined according to technical approaches covering the whole range from allowed minimum to maximum.
Distribution of rotation parameters can be Gaussian or biological.

In addition to the rotation period the rotation angle, the rotation speed and the resting periods can be varied. In order to adjust for variable rotation angles, speed and resting times, a mean product of angle and resting period etc can be defined, that needs to be kept constant. For example:
While rotation angle randomly varies about a given rotation angle, resting periods are adjusted to keep the product of angle and time approximately constant at a given rotation speed.
While rotation angle randomly varies about a given rotation angle, rotation speed is adjusted to keep the product of angle and speed approximately constant while no resting period is applied.

FIG. 2 shows a first example of a position actuator in a horizontal position representing the initial position. The schematic drawing depicts the patient 201 lying in the supine position. As defined in medical imaging, the patient is looked at from the feet, thus the right lung (R) is on the left hand side of FIG. 2, and the left lung (L) is on the right hand side of FIG. 2, while the heart (H) is located centrally and towards the front.

It should be noted in this connection that the methods according to the invention can be equally well applied to patients lying in the prone position.

The patient is lying on a supporting surface 202, which covers three air-cushions 203, 204 and 205. These air-cushions, being mounted to the fixed frame 206 of the nursing bed, are inflated in this horizontal position of the nursing bed with a medium air pressure. The air pressure of the air-cushions 203, 204 and 205 can be adjusted by a control unit either by pumping air into an air-cushion or by deflating an air-cushion. Obviously, other fluids than air could be used as well.

Changing the air pressure in the air-cushions 203, 204 and 205 in a particular fashion leads to a rotation of the supporting surface 202 and hence to a rotation of the artificially ventilated lung. By simultaneous measurements of the rotation angle of the artificially ventilated lung, i.e. through an attached position sensor at the patient's thorax, the rotation angle of the artificially ventilated lung can be adjusted to defined positions. Alternatively, a defined lung position can be reached by a predetermined step size of the position actuator, i.e. a predetermined air pressure within each air-cushion.

FIG. 3 shows the first example of the position actuator in an angulated position resulting from a specific setting of the air pressures in the air-cushions. Compared to FIG. 2, in this particular example the air pressure of the air-cushion 303 has been lowered, the air pressure of the air-cushion 304 has not been changed, and the air pressure of the air-cushion 305 has been raised.

This results in a rotation of the supporting surface 302 and thus in a rotation of the artificially ventilated lung. Noticeably, the frame 306 of the nursing bed remains in its horizontal position.

FIG. 4 shows a second example of a position actuator in a horizontal position representing the initial position. The schematic drawing depicts the patient 401 lying in the supine position as defined in the description of FIG. 2.

The patient is lying on a supporting surface 402, which is attached to the frame 403 of the nursing bed. The frame 403 can be rotated by a motor which represents the position actuator according to signals received from a control unit. A rotation of the frame 403 results directly in a rotation of the patient and hence the artificially ventilated lung. By simultaneous measurements of the rotation angle of the artificially ventilated lung, i.e. through measurements of the rotation angle of the frame 403, the rotation angle of the artificially ventilated lung can be adjusted to defined positions. Alternatively, a defined lung position can be reached by a predetermined step size of the position actuator, i.e. performing a predetermined number of steps using a step motor.

FIG. 5 shows the second example of a position actuator in an angulated position, resulting from a specific setting of the position actuator. In this particular setting of the position actuator the left lung of the patient is elevated. The supporting surface 502 and the frame 503 of the nursing bed are both rotated.

FIG. 6 shows a schematic monitoring screen for the method for controlling at least one ventilation pressure. Displayed are both the input of the artificial ventilation system in form of the PIP and the PEEP as well as an example of a physiological output information of the patient in form of the on-line SpO₂ signal. The SpO₂ signal represents the oxygen saturation level. The values of the PIP, the PEEP, and SpO₂ are plotted in a circular coordinate system over the rotation angle of the artificially ventilated lung. The rotation angle is depicted in FIG. 6 through the dashed lines for values of −45°, 0°, and 45°. The values for the PIP, the PEEP, and SpO₂ can be obtained from the graph using an axis perpendicular to the axis of the particular rotation angle.

As can be seen from FIG. 6, when the nursing bed turns the patient towards a negative rotation angle, the value of the SpO₂ signal increases substantially, whereas the value of the SpO₂ signal decreases, when the patient is turned towards a positive rotation angle.

This variation of the SpO₂ signal relates to constant values of the PIP and the PEEP. Without changing at least one of the airway pressures the evaluation of the SpO₂ signal of the patient during a rotation would only represent a diagnostic goal. Therefore, FIGS. 7-10 represent the effects of controlling at least one ventilation pressure on a physiological output information.

FIG. 7 shows an alveolar recruitment maneuver during kinetic rotation therapy Before the recruitment maneuver starts at 0° rotation angle, the PEEP is adjusted above the expected alveolar closing pressure (depending on the lung disease between 15 and 25 cmH₂O). The PIP is set sufficiently high above the PEEP to ensure adequate ventilation.

During the recruitment maneuver the PIP is stepwise increased such that as many lung units as possible are re-opened, while at the same time the PEEP is maintained at a level to keep the newly recruited lung units open. The recruitment is applied towards the maxima of the positive and the negative rotation amplitudes where the respective upper lung is relieved from almost all superimposed pressures. Therefore, each lung is opened separately while it is moved into the upward position.

For example the stepwise increase of the PIP can start 5-20 breaths prior to reaching the maximum rotation angle and the PIP reaches its maximum value (depending on the lung disease between 45 and 65 cmH₂O) at the maximum rotation angle. Having crossed the maximum rotation angle the PIP is decreased within 5-20 breaths to its initial value.

After each lung has been recruited separately (by rotating the patient to both sides) in the above manner, PIP can be adjusted for each lung separately to maintain adequate ventilation.

FIG. 8 shows the titration process after a successful alveolar recruitment maneuver has been performed during kinetic rotation therapy.

Due to the hysteresis behaviour of the lung, the values obtained for the PIP and for the PEEP during the alveolar recruitment maneuver are too high to further ventilate the lung with these airway pressures once the lung units have been recruited. Thus they need to be reduced systematically during the titration process. The goal is to obtain the minimum values for the PEEP for specific rotation angles that would just keep all lung alveoli open. For further ventilation the PEEP can be set slightly above these values and the PIP can be adjusted according to the desired tidal volume.

As shown in FIG. 8A the PIP and the PEEP are reduced, typically in periods of one step-wise reduction per minute, towards both maxima of the rotation amplitude. The titration process begins with decreasing the PIP and/or the PEEP when rotating the artificially ventilated lung towards positive rotation angles (procedure 1). When the artificially ventilated lung is returned to the initial position, i.e. 0° rotation angle, the PIP and the PEEP are set to their initial values. The PIP and/or the PEEP are reduced again once the artificially ventilated lung is rotated towards negative rotation angles (procedure 2). As an example of a physiological feedback parameter the oxygen saturation signal SpO₂ is shown in FIG. 8A as a dashed line. The oxygen saturation remains constant during the entire rotation cycle (procedure 1+procedure 2), indicating that no significant collapse occurred. Thus the titration process has to continue.

In order to increase the likelihood of a collapse of lung units, each subsequent rotation cycle starts with lower values for the PIP and for the PEEP. FIG. 8B represents a further rotation cycle of the titration process. The oxygen saturation signal SpO₂ remains again constant during the rotation cycle shown in FIG. 8B, indicating that the lowest values of the PEEP reached at the maximum rotation angles are still too high to result in a significant collapse of lung units.

A further reduction of the PIP and the PEEP has been performed before commencing the next rotation cycle as shown in FIG. 8C. When turning the patient to positive rotation angles and reducing the PEEP (procedure 1), the oxygen saturation signal SpO₂ shows a variation in form of a reduction. Once this variation has been identified, no further reductions of the airway pressures are performed. The PEEP corresponding to the point when the variation of the oxygen saturation signal SpO₂ has been identified represents the collapse pressure for the particular rotation angle. The titration process for positive rotation angles is finished.

When turning the patient back towards the initial position, i.e. 0° rotation angle, the PIP and the PEEP are set to their original values. The oxygen saturation signal SpO₂ recovers to its initial value. As indicated in FIG. 8C a hysteresis effect is usually present.

When turning the patient to negative rotation angles the PIP and/or the PEEP are reduced in order to identify the collapse pressure for negative rotation angles (). The oxygen saturation signal SpO₂ remains constant, indicating that the value of the PEEP reached at the maximum negative rotation angle is still too high to result in a significant collapse of lung units. Consequently, the titration process at negative rotation angles has to continue.

A further rotation cycle starting once more with lower values for the PIP and for the PEEP is shown in FIG. 8D. As indicated, collapse pressures for positive and for negative rotation angles can be identified according to the procedure of FIG. 8C. The collapse pressure for the positive rotation angle, corresponding to the value already obtained in FIG. 8C, is lower than the collapse pressure for the negative rotation angle.

After having identified the collapse pressures for positive and negative rotation angles a recruitment maneuver according to FIG. 7 needs to be carried out in order to re-open lung units which collapsed during the titration process. As mentioned before, such a re-opening procedure can become necessary already during the titration process once the collapse pressure for one side has been identified. This is the case, if, due to a hysteresis behaviour of the lung, signs of lung collapse continue to be present when the patient is turned back to 0° and the PEEP is raised to its previous setting when at 0°.

Once the lung is fully recruited again, the PEEP levels are set for the positive and negative rotation angles separately according to the collapse pressures as identified before. A safety margin of i.e. 2 cmH₂O is added to each collapse pressure. Eventually, the PIP can be adjusted according to the desired tidal volume.

FIG. 9 shows an artificial ventilation of a lung by controlling the PIP and the PEEP in accordance with the rotation angle. Based on the collapse pressures for positive and for negative rotation angles, as identified according to FIG. 8, a curve for the PEEP as a function of the rotation angle can be established. The shape of the curve, having in this particular example a smooth curvature, can be chosen freely, provided a safety margin is realized in order to keep the PEEP above the corresponding collapse pressure. The curve of the PIP as a function of the rotation angle follows directly from the corresponding PEEP value and the desired tidal volume.

Controlling the PIP and the PEEP as a function of the rotation angle in this way leads to an optimal ventilation of the lung. The oxygen saturation signal SpO₂ remains constant during the rotation cycle while at the same time, due to the lowest possible values for the PIP and the PEEP, no lung over-distension is present and the desired tidal volume is achieved.

FIG. 10 shows a schematic monitoring screen when controlling the PIP and the PEEP during the rotation cycle according to FIG. 9. The presentation of the PIP, the PEEP, and the SpO₂ with respect to the rotation angle is identical to that of FIG. 6.

By controlling the PIP and the PEEP according to the rotation angle it is possible to keep the oxygen saturation signal SpO₂ constant during a rotation cycle. This is in contrast to FIG. 6 where the oxygen saturation signal SpO₂ decreased with increasing rotation angles, i.e. due to the collapse of lung units. This collapse is prevented within the artificial ventilation shown in FIG. 10 by controlling the PIP and the PEEP accordingly.

FIG. 11 shows the measurements of paO₂, paCO₂, and pHa during the kinetic rotation therapy. As it can be seen, paO₂ improves continuously during the kinetic rotation therapy. The rotation period was switched during kinetic rotation therapy from 8 to 16 rotation periods per hour. Having a mean ventilation frequency of 10 to 40 breaths per minute this results in 50 to 250 breaths per rotation period.

The schematic drawing of FIG. 11 is derived from an original on-line blood gas registration by the blood gas analyzer Paratrend (Diametrics, High Newcombe, UK) of a patient suffering from adult respiratory distress syndrome (ARDS) who is treated in a nursing bed employing a Servo 300 ventilator (Siemens Elema, Solna, Sweden). Rotation angles ranged from −62° to +62°0. While the mean paO₂ improves continuously during the kinetic rotation therapy, paO₂ also oscillates around a mean value resulting from turning the patient from one side to the other. The oscillation reflects the fact that artificially ventilating the patient at one side seems to be more effective for improving paO₂ than artificially ventilating the patient at the other side.

Without additional data the blood gas analysis does not give any information about the relationship between the rotation angle, the ventilator settings and their final effect on gas exchange. The registration shows, however, the influence of the rotation period on the mean paO₂ and its oscillations. As stated above, in this particular example the rotation period was switched from 8 to 16 rotation periods per hour. While paO₂ increased, the amplitude of the oscillations was considerably reduced, indicating that the individual and time dependent influences of the sick lung and the normal lung are minimized.

It becomes obvious that a link between at least two of the factors rotation angle, ventilator settings, and physiological output variable is needed.

FIG. 12 shows a measurement of the compliance during the kinetic rotation therapy. As expected, the compliance improves during the kinetic rotation therapy. As explained above, the ventilation parameters are adapted accordingly. It should be noted, that the range of the rotation angle shown in FIG. 12 represents only one example. Higher values for the rotation angle, i.e. ±90° or even more, can be chosen if required.

The compliance is displayed as a function of the rotation angle. When the patient is turned towards +62° rotation angle (following the bold line from its beginning at 0° rotation angle) the compliance decreases to almost half of its initial value at 0° rotation angle. As the patient is turned back to the initial position at 0° rotation angle, the compliance increases even beyond the initial value and continues to improve as the patient is turned towards negative rotation angles. The compliance reaches its temporary maximum at −62° rotation angle. As the patient is turned back to the initial position at 0° rotation angle, the compliance decreases continuously but remains significantly above the value at the previous zero-degree-transition. As kinetic rotation therapy continues, the compliance values follow a similar pattern as described, however, the incremental improvements per rotation cycle become smaller and it is apparent that a certain saturation of the therapeutic effect has been reached. For the sake of an even further improvement of the lung function, a superimposed active therapeutic intervention like an alveolar recruitment maneuver by means of a ventilator should be applied.

1. A recording method to record a status of an artificially ventilated lung of a patient in accordance with a plurality of lung positions, the patient lying in a nursing bed and a position of the artificially ventilated lung is moveable by a position actuator, comprising the steps of:
a) moving the artificially ventilated lung by the position actuator to a defined lung position,
b) determining the status of the artificially ventilated lung, and
c) recording the status of the artificially ventilated lung in accordance with the defined lung position.

2. The recording method of claim 1, wherein the nursing bed is rotatable around its longitudinal axis and wherein the position actuator is a motor rotating the nursing bed around its longitudinal axis.

3. The recording method of claim 1, wherein the position actuator comprises air cushions provided underneath the patient.

4. The recording method of claim 1, wherein the defined lung position is reached by a predetermined step size of the position actuator.

5. The recording method of claim 1, wherein the defined lung position is reached in accordance with a feed back signal of a position sensor measuring the actual lung position.

6. The recording method of claim 1, wherein the status of the artificially ventilated lung includes a measure of a regional or a global information on lung morphology and/or lung function.

7. The recording method of claim 1, wherein the status of the artificially ventilated lung includes a measure of the functionality with regard to the global gas exchange of the lung.

8. The recording method of claim 1, wherein the determined status of the artificially ventilated lung includes recording by a computer in accordance with the corresponding defined lung position.

9. The recording method of claim 1, wherein the steps a), b), and c) are repeated with a predetermined differential step size of the position actuator until the status of the artificially ventilated lung has been determined over a predetermined range of lung positions.

10. A controlling method to control at least one ventilation pressure of an artificial ventilator for ventilating an artificially ventilated lung of a patient in accordance with a plurality of lung positions, the patient lying in a nursing bed and the position of the artificially ventilated lung is moveable by a position actuator, comprising the steps of:
a) obtaining lung status information which is based on at least two supporting points of a first status of the artificially ventilated lung in accordance with a first lung position and a second status of the artificially ventilated lung in accordance with a second lung position,
b) moving the artificially ventilated lung by the position actuator to a defined lung position,
c) controlling of at least one ventilation pressure in accordance with the defined lung position and in accordance with the lung status information related to said defined lung position.

11. (canceled)

12. The controlling method of claim 10, wherein the lung status information is interpolated between the supporting points in accordance with the difference between two neighboring supporting points.

13. The controlling method of claim 10, wherein at least one ventilation pressure is controlled such that the lung status information yields a homogeneous distribution over a plurality of lung positions.

14. A positioning method to control the change of a position of an artificially ventilated lung of a patient, the patient lying in a nursing bed and the position of the artificially ventilated lung is changeable by a corresponding position actuator, comprising the steps of:
a) providing a periodical controlling signal having a distribution of a plurality of position periods and/or of a plurality of amplitudes,
b) controlling the position actuator by said periodical controlling signal.

15. The positioning method of claim 14, wherein the distribution is compiled via a user's interface on the basis of a given set of periodical controlling signals.

16. The positioning method of claim 14, wherein the distribution is compiled in accordance with lung status information which is based on at least two supporting points of a first status of the artificially ventilated lung in accordance with a first lung position and a second status of the artificially ventilated lung in accordance with a second lung position.

17. A recording apparatus to record a status of an artificially ventilated lung of a patient lying in a nursing bed in accordance with a plurality of lung positions, comprising:
a) a position actuator to move the artificially ventilated lung to a defined lung position,
b) determining means to determine the status of the artificially ventilated lung, and
c) recording means to record the status of the artificially ventilated lung in accordance with the defined lung position.

18. The recording apparatus of claim 17, wherein the nursing bed is rotatable around its longitudinal axis and wherein the position actuator is a motor rotating the nursing bed around its longitudinal axis.

19. The recording apparatus of claim 17, wherein the position actuator comprises air cushions provided underneath the patient.

20. The recording apparatus of claim 17, wherein the defined lung position is reached by a predetermined step size of the position actuator.

21. The recording apparatus of claim 17, wherein the defined lung position is reached in accordance with a feed back signal of a position sensor measuring the actual lung position.

22. The recording apparatus of claim 17, wherein the status of the artificially ventilated lung is a measure of a regional or a global information on lung morphology and/or lung function.

23. The recording apparatus of claim 17, wherein the status of the artificially ventilated lung is a measure of the functionality with regard to the global gas exchange of the lung.

24. The recording apparatus of claim 17, wherein the determined status of the artificially ventilated lung is recorded by a computer in accordance with the corresponding defined lung position.

25. The recording apparatus of claim 17, wherein a predetermined differential step size is applied repeatingly to the position actuator until the status of the artificially ventilated lung has been determined over a predetermined range of lung positions.

26. A controlling apparatus to control at least one ventilation pressure of an artificial ventilator for ventilating an artificially ventilated lung of a patient lying in a nursing bed in accordance with a plurality of lung positions, comprising:
a) means for obtaining lung status information which is based on at least two supporting points of a first status of the artificially ventilated lung in accordance with a first lung position and a second status of the artificially ventilated lung in accordance with a second lung position,
b) a position actuator to move the artificially ventilated lung to a defined lung position,
c) means for controlling of at least one ventilation pressure in accordance with the defined lung position and in accordance with the lung status information related to said defined lung position.

27. The controlling apparatus of claim 26, wherein the lung status information is obtained by using the recording apparatus according to claim 25.

28. The controlling apparatus of claim 26, wherein the lung status information is interpolated between the supporting points in accordance with the difference between two neighbouring supporting points.

29. The controlling apparatus of claim 26, wherein at least one ventilation pressure is controlled such that the lung status information yields a homogeneous distribution over a plurality of lung positions.

30. A positioning apparatus to control the a change of a position of an artificially ventilated lung of a patient lying in a nursing bed, comprising:
a) a position actuator for changing the position of the artificially ventilated lung,
b) means for providing a periodical controlling signal having a distribution of a plurality of position periods and/or of a plurality of amplitudes, and
c) means for controlling the position actuator by said periodical controlling signal.

31. The positioning apparatus of claim 30, wherein the distribution is compiled via a user's interface on the basis of a given set of periodical controlling signals.

32. The positioning apparatus of claim 30, wherein the distribution is compiled in accordance with lung status information which is based on at least two supporting points of a first status of the artificially ventilated lung in accordance with a first lung position and a second status of the artificially ventilated lung in accordance with a second lung position.

33. The controlling method of claim 10, wherein the lung status information is obtained by a recording method, the recording method to record a status of an artificially ventilated lung of a patient in accordance with the plurality of lung positions, the recording method comprising the steps of:
a) moving the artificially ventilated lung by the position actuator to a defined lung position,
b) determining the status of the artificially ventilated lung,
c) recording the status of the artificially ventilated lung in accordance with the defined lung position, and
repeating the steps a), b), and c) with a predetermined differential step size of the position actuator until the status of the artificially ventilated lung has been determined over a predetermined range of lung positions.
